(19) United States
(12) Patent Application Publication
(10) Pub. No.: US 2007/0044810 A1
(43) Pub. Date: Mar. 1, 2007

(54) Method of enhanced drug application

(75) Inventors: Jose E. Ramirez, Trumbull, CT (US); Joseph R. Faryniarz, Middlebury, CT (US)

Correspondence Address: CARTER, DELUCA, FARRELL & SCHMIDT, LLP, 445 BROAD HOLLOW ROAD, SUITE 225, MELVILLE, NY, 11747, US

(73) Assignee: JR CHEM, LLC

(21) Appl. No.: 11/476,954

(22) Filed: Jun. 28, 2006

Related U.S. Application Data
(60) Provisional application No. 60/695,026, filed on Jun. 29, 2005.

(51) Int. Cl. A61B 19/00

(52) U.S. Cl. 128/898

(57) ABSTRACT

Methods of treating skin in need thereof are disclosed including applying a drug or active agent or formulation thereof to the skin of a user and applying a sealer thereto. The methods are useful in actively enhancing penetration of the actives immediately upon application to skin, sealing the skin surface to prevent removal of the drug or active agent, holding the drug or active agent in a reservoir film, and/or enhancing long term penetration of the drug or active agent.

### RELATED APPLICATION

This Application claims priority benefit of U.S. Provisional Application No. 60/695,026 filed Jun. 29, 2005, herein incorporated by reference in its entirety.

### BACKGROUND

1. Technical Field

This disclosure relates to application of active agents and, more specifically to a treatment regimen that improves percutaneous absorption of an active agent by use of a sealer. The method generally has two steps including applying a suitable active agent to the skin such as a drug, and applying a sealer to the treated skin. The method, inter alia, improves the penetration of an active agent into the skin.

2. Background of Related Art

The integumentary system includes the skin and all the structures associated with skin such as hair, nails, sweat glands and oil glands. The functions of the integumentary system include, inter alia, providing a protective barrier for the body to prevent the entry of potentially harmful things. Unfortunately, the protective barrier function of the skin may slow and/or prevent the penetration of active agents such as drugs applied to the skin and/or reduce the efficacy of topically applied active agents. Furthermore, during skin treatment, the problematic nature of the protective barrier function of the skin may be compounded by other factors such as characteristics of the active agents themselves and excipients combined therewith during the formulation processes, the condition of the skin prior to application of the active agent, the amount of time the active agent remains in contact with the skin, and/or the diseased or injured state of the skin.

Although treatments are known for applying active agents such as drugs to skin, known treatments are problematic in that they do not demonstrate immediate penetration of drug or active agent, and results may vary from patient to patient. Furthermore, no one treatment, if ever, obtains maximum benefit for every patient. As a result, some individuals may have slower active agent penetration and an increased risk for complications during healing. Accordingly, novel skin treatments are continuously sought after to help enhance penetration of the active agents immediately upon application to skin and there remains room for improvement in skin treatment regimens to enhance active agent penetration.

What are needed are new skin care compositions and methods of applying an active agent such as a drug to skin that will actively enhance penetration of the actives immediately upon application to skin; prevent removal of the active agent; hold the drug or active agent in a reservoir film; and/or enhance long term penetration of the drug.

### SUMMARY

This disclosure relates to application of active agents and, more specifically to a treatment regimen that improves percutaneous absorption of an active agent by use of a sealer. Applying a sealer to an area of skin treated with a drug or active agent will, among other things, actively enhance penetration of the actives immediately upon application to skin, seal the skin surface to prevent removal of the drug or active agent, hold the drug or active agent in a reservoir film, and/or enhance long term penetration of the drug. Accordingly, active agents such as topically applied drugs and formulations containing them may be used in combination with a sealer to treat undesirable skin conditions.

Skin having one or more undesirable skin conditions is treated in accordance with the present disclosure by topically treating skin with one or more active agents, followed by the immediate application of a sealer to the treated skin. For example, compositions containing antifungal and/or moisturizer can be directly applied to skin in need of treatment, prior to the application of a sealer. Post-treatment by application of a sealer, among other things, increases the penetration and/or efficacy of the active agent.

In addition, dermatological treatment regimens in accordance with the present disclosure may improve characteristics of a user's skin. The regimens include the repeated topical application of one or more active agents, and the repeated topical application of a sealer.

These and other aspects of this disclosure will be evident upon reference to the following detailed description.

### DETAILED DESCRIPTION OF PREFERRED EMBODIMENTS

The present disclosure relates to compositions and methods for application of active agents to skin. The method includes applying a predetermined amount of active agent such as a drug to an area of skin in need thereof and applying a sealer to the treated area. Sealer is applied over the drug or active agent to an area of skin in need of treatment. In embodiments, the sealer dries on the skin surface sealing the remaining drug in place. The application of the a sealer actively enhances penetration of the active agents immediately upon application to skin, seals the skin surface to prevent removal of the active agents, and/or enhances long term penetration of the active agent. In embodiments, the sealer holds active agent such as a drug in a reservoir film.

In embodiments, a treatment regimen in accordance with this disclosure includes the sequential use of at least two products; namely at least one active agent or drug and/or mixtures of formulations such as compositions containing active agents or drugs, and at least one sealer. In one embodiment, active agents may be combined with a blender composition. Here, the treatment includes the step of pre-mixing an active agent with a blending composition to form a pre-mix; and then applying the pre-mix to the skin. In embodiments, the percutaneous absorption of the active agent in increased compared to the application of the active agent or pre-mix without a sealer.

Suitable active agents may be used either alone, or in combination with a composition and/or formulation. For example, the active agent such as a drug may be in solution form, a topical formulation characterized as a clear facial serum, a topical formulation characterized as a stick, or an emulsion such as a blender composition.

Non-limiting examples of active agents such as drugs are listed below. Although suitable active agents including drugs are categorized in various classes, this classification is not intended to limit the active agents in any way to only those active agents belonging to the categories herein mentioned.

Antimicrobial Actives

Antimicrobials suitable for use in accordance with the present disclosure include all antibiotics, antimicrobial agents and antimicrobial peptides. Antibiotics that may be used include, inter alia, dermatologically acceptable salts of tetracyclin and tetracycline derivatives, gentamycin, kanamycin, streptomycin, neomycin, capreomycin, lineomycin, paromomycin, tobramycin, erythromycin, triclosan, octopirox, parachlorometa xylenol nystatin, tolnaftate, miconazole hydrochloride, chlorhexidine gluconate, chlorhexidine hydrochloride, methanamine hippurate, methanamine mandelate, minocycline hydrochloride, clindamycin, cleocin, b-lactam derivatives such as aminopenicillin and mixtures thereof. In embodiments, chlorhexidine gluconate and triclosan are suitable for use herein.

In embodiments, antimicrobial agents that may be used in accordance with the present disclosure include for example benzoyl peroxide and salicylic acid.

In embodiments, antimicrobial peptides useful herein are for example magainin, nicin and cecropin.

Anti-Acne Actives

Anti-acne actives suitable for use in accordance with the present disclosure include without limitation tretinoin, keratolytic agents including lactic acid, pyruvic acid, salicylic acids, urea and N-acetylcysteine, retinoids, and retinoid analogs such as tretinoin, cis and trans retinoic acid, retinol and retinol palmitate, isotretinoin-13-cis-retinoic acid, antibiotics and antimicrobial agents such as tetracycline, erythromycin, minocycline, clindamycin, trimethoprim-sulphamethazole and anti-microbial peptides (nicin, for example), steroids, such as hydrocortisone, gamma-linolenic acid and mixtures thereof. Further anti-acne actives that may be used include without limitation benzoyl peroxide, alpha and beta hydroxy acids, sulfacetamide and sulfur, and mixtures thereof. In embodiments, salicylic acid, benzoyl peroxide and retinoids are suitable for use herein.

In embodiments, benzoyl peroxide serums are suitable for use in accordance with the present disclosure. Suitable benzoyl peroxide serums are described in U.S. application Ser. No. 11/373,538 filed Mar. 10, 2006 entitled Benzoyl Peroxide Compositions and Methods of Use (herein incorporated by reference in its entirety).

Anti-Psoriasis Actives

Anti-psoriasis actives suitable for use in accordance with the present disclosure include without limitation salicylic acid, mometasone furoate, steroids including corticosteroids such as cortisone and oluxclobetasol propionate, 5-fluorouracil, epinephrine, anthralin, vitamin D3 analogs, such as calcipotriene, methotrexate, masprocol, trimethaxate gluconate, retinoids, cyclosporin, paclitaxel, 5-amino levulinic acid, bergasol, tin-ethyl etio purpurin, benzoporphyrin derivatives, antibodies, such as ABX-IL8 antibody, CD11a monoclonal antibody and ICM3 monoclonal antibody, enzyme inhibitors, including tryptase inhibitor and phospholipase A-2 inhibitors, angiogenesis blocking agents, and T-cell blocking agents, and mixtures thereof.

Anti-Eczema Actives

Anti-eczema actives suitable for use in accordance with the present disclosure include urea, evening primrose oil, plant extracts, hydrocortisone, an immunomodulator, tar combined with fatty acids obtained from banana, and mixtures thereof.

Topical Anesthetic Actives

Topical anesthetic actives suitable for use in accordance with the present disclosure include tetracaine, lidocaine, editocaine, bupivacaine, pramoxine, and mixtures thereof.

Anti-Inflammatory Actives

Anti-inflammatory actives suitable for use in accordance with the present disclosure include steroidal actives such as hydrocortisone as well as non-steroidal actives including propionic derivatives, acetic acid derivatives, biphenylcarboxylic acid derivatives, fenamic acid derivatives, and oxicams. Examples of anti-inflammatory actives include without limitation acetaminophen, oxaprozin, pranoprofen, benoxaprofen, bucloxic acid, elocon, and mixtures thereof.

Vitamin Actives

Vitamin actives suitable for use in accordance with the present disclosure include vitamin A and derivatives, including retinoic acid, retinyl aldehyde, retin A, retinyl palmitate, adapalene, and beta-carotene, vitamin B (panthenol, provitamin B5, panthenic acid, vitamin B complex factor), vitamin C (ascorbic acid and salts thereof) and derivatives such as ascorbyl palmitate, vitamin D including calcipotriene (a vitamin D3 analog), vitamin E including its individual constituents alpha-, beta-, gamma-, delta-tocopherol and cotrienols and mixtures thereof and vitamin E derivatives including vitamin E palmitate, vitamin E linolate and vitamin E acetate, vitamin K and derivatives, vitamin Q10 (ubiquinone), and mixtures thereof.

Protein Actives

One class of actives suitable for use in accordance with the present disclosure are proteins and peptides. In principle, any desired protein or peptide and oil bodies comprising these proteins (including recombinant proteins) may be applied in accordance with the present disclosure. Proteins and peptides which may be used in accordance with the present disclosure include enzymes such as proteases (e.g. bromelain, papain, collagenase, elastase), lipases (e.g. phospholipase C), esterases, glucosidases, exfoliating enzymes, antibodies and antibody derived actives, such monoclonal antibodies, polyclonal antibodies, single chain antibodies and the like, reductase, oxidases, peptide hormones, natural structural skin proteins, such as elastin, collagen, reticulin and the like, growth factors such as platelet derived growth factor (PDGF) and epidermis derived growth factor (EGF), anti-oxidants such as superoxide dismutase, catalase and glutathione, free-radical scavenging proteins, DNA-repair enzymes, for example T4 endonuclease 5 and P53, antimicrobial peptides, such as magainin and cecropin, a milk protein, a silk protein or peptide, and any active fragments, derivatives of these proteins and peptides, and mixtures thereof.

Antifungal Actives

In embodiments, antifungal active agents may be used in amounts sufficient to minimize, and/or eliminate fungus on the skin of a user. Any antifungal agent can be used provided it can be topically applied to the skin of the user. Non-limiting examples of antifungal agents include topically applied allylamines such as naftifine hydrochloride, topically applied azoles such as clotrimazole, econazole, ketoconazole, miconazole nitrate, oxiconazole nitrate, sertaconazole nitrate, and sulconazole nitrate, and other antifungal agents such as butenafine hydrochloride 1%, ciclopirox, clotrimazole-betamethasone, haloprogrin, nystatin, and combinations thereof.

Miscellaneous Active Agents

Further active agents suitable for use in accordance with the present disclosure include an amino acid and amino acid derivative, an insect repellant, a fungicide, an anti-viral agent (such as acyclovir), an anti-cancer agent, a plant extract, an anti-hemorrhoid compound, an anti-dandruff compound, a hair-growth stimulating compound, a hair loss stimulating compound, a nucleic acid (DNA, RNA and derivatives), an anti-scabies agent (such as permethrin), an anti-wart agent (such as podophyllotoxin), a copper-zinc salt such as copper-zinc malonate, and mixtures thereof.

Other active agents suitable for use in accordance with the present disclosure include multifunctional acids salts, such as copper-zinc salts of multifunctional organic acids and formulations containing them. Non-limiting examples of such salts and formulations containing them include copper-zinc malonate, copper-zinc malonate solution, or other copper-zinc malonate cream or other formulation.

In embodiments, organic peroxide actives and formulations containing them are suitable for use in accordance with the present disclosure. Suitable organic peroxide actives and formulations are described in U.S. application Ser. No. 11/372,958 entitled Stable Organic Peroxide Compositions (herein incorporated by reference in its entirety).

The active agents or drugs may be combined with numerous ingredients to form products to be applied to the skin, or other tissues of humans or other mammals. Such products may include a dermatologically or pharmaceutically acceptable carrier or diluent, vehicle or medium, for example, a carrier, vehicle or medium that is compatible with the tissues to which they will be applied. The term “dermatologically or pharmaceutically acceptable” as used herein, means that the compositions or components thereof so described are suitable for use in contact with these tissues or for use in patients in general without undue toxicity, incompatibility, instability, allergic response, and the like. In embodiments, compositions in accordance with the present disclosure can contain any ingredient conventionally used in cosmetics and/or dermatology.

As an illustrative example, compositions can be formulated to contain active agent in an amount of about 0.001 to about 20% by weight of the total composition. In embodiments, products can be formulated to contain active agent in an amount of about 0.05 to about 10% by weight of the total composition. In other embodiments, the active agent is present in an amount of about 0.1 to about 5.0% by weight of the total composition. In such embodiments, the active agents present may be in a pharmaceutically acceptable salt form.

In embodiments, products containing active agents in accordance with the present disclosure can be in the form of solutions, emulsions (including microemulsions), suspensions, creams, fluid cream, oils, lotions, gels, powders, sticks, or other typical solid or liquid compositions used for treatment of undesirable skin conditions. Such compositions may contain, in addition to the active agents in accordance with this disclosure, other ingredients typically used in such products, such as other active cosmetic substances such as retinol, retinol derivatives, allantoin, tocopherol, tocopherol derivatives, niacinamide, phytosterols, isoflavones, panthenol, panthenol derivatives, bisabolol, farnesol, and combinations thereof, other active drug substances such as corticosteroid, metronidazole, sulfacetamide, sulfur, and combinations thereof, antioxidants, antimicrobials, coloring agents, detergents, dyestuffs, emulsifiers, emulsifying wax, emollients, fillers, fragrances, gelling agents, hydration agents, moisturizers, odor absorbers, natural or synthetic oils, penetration agents, powders, preservatives, solvents, surfactants, thickeners, viscosity-controlling agents, water, distilled water, waxes, and optionally including anesthetics, anti-itch actives, botanical extracts, conditioning agents, darkening or lightening agents, glitter, humectant, mica, minerals, polyphenols, phytomedicinals, silicones or derivatives thereof, skin protectants, sunblocks, vitamins, and mixtures or combinations thereof. Such compositions may also contain, in addition to the active agents in accordance with this disclosure, one or more: fatty alcohols, fatty acids, organic bases, inorganic bases, wax esters, steroid alcohols, triglyceride esters, phospholipids, polyhydric alcohol esters, fatty alcohol ethers, hydrophilic lanolin derivatives, hydrophilic beeswax derivatives, cocoa butter waxes, silicon oils, pH balancers, cellulose derivatives, hydrocarbon oils, or mixtures and combinations thereof.

In embodiments, product forms can be formulated to contain humectant in an amount of about 1% to about 15% by weight of the total composition. For example glycerine can be added to the composition in an amount of about 1% to about 15% by weight of the total composition. In particular embodiments, glycerine can be added to the composition in an amount of about 1% to about 5% by weight of the total composition.

In embodiments, product forms can be formulated to contain solvent in an amount of about 1% to about 45% by weight of the total composition. For example petroleum derivatives such as propylene glycol can be added to the composition in an amount of about 1% to about 45% by weight of the total composition. In particular embodiments, propylene glycol, polyethylene glycol, ethoxy diglycol can be added to the composition in an amount of about 15% to about 30% by weight of the total composition.

In embodiments, product forms can be formulated to contain water in an amount of about 40% to about 99% by weight of the total composition. For example distilled water can be added to the composition in an amount of about 40% to about 99% by weight of the total composition. In particular embodiments, distilled water can be added to the composition in an amount of about 65% to about 80% by weight of the total composition.

In embodiments, active antifungal agent such as nystatin may be used in a cream and applied to the skin of a user. For example, nystatin cream can be applied around the toes of a user to treat an antifungal infection. In embodiments, nystatin cream, such as cream further illustrated in Example 3 below, may be applied to the skin of a user prior to the application of a sealer.

In embodiments, active agent such as moisturizer may be applied to the skin of a user prior to the application of a sealer. For example, moisturizing cream can be applied around the heel of a user to treat cracks.

The treatments in accordance with the present disclosure also include applying a sealer to the area of skin in need thereof. The application of the sealer is useful in actively enhancing penetration of the actives immediately upon application to skin, sealing the skin surface to prevent removal of the drug or active agent, holding the drug or active agent in a reservoir film, and/or enhancing long term penetration of the drug or active agent.

The sealer may be made of a number of constituents including one or more solvents, penetration aids, and polymer substances including film-forming substances, and combinations thereof.

Non-limiting examples of suitable polymers for use in accordance with the sealer of the present disclosure include natural polymers, acrylic resins, silicones, celluloses, alkyd resins, carboxyvinyl polymers, vinylpyrrolidone-based polymers, type A methacrylic acid copolymer such as Eudragit L 100 brand copolymer, type B methacrylic acid copolymer such as Eudragit S 100 brand copolymer, and combinations thereof.

Non-limiting examples of suitable acrylic resins for use in accordance with the sealer of the present disclosure include polyacrylic acid, poly(methyl acrylate), poly-(ethyl acrylate), poly(butyl acrylate), polyacrylamide, poly(N-isopropylacrylamide), ammonium polyacrylate, sodium polyacrylate), crosslinked sodium polyacrylate, polymethacrylic acid, poly(methyl methacrylate, poly-(ethyl methacrylate), poly(butyl methacrylate), polymethacrylamide, sodium methacrylate, acrylic acid-styrene-ammonium methacrylate copolymers, acrylic acid-styrene copolymers, acrylic acid-methacrylamide copolymers, alkyl acrylate-styrene copolymers, alkyl acrylate copolymers, ethyl acrylate-acrylamide-acrylic acid copolymers, ethyl acrylate-butyl acrylate copolymers, ethyl acrylate-ethyl methacrylate copolymers, ethyl acrylate-methyl methacrylate-acrylic acid copolymers, ethyl acrylate-methyl methacrylate copolymers, ethyl acrylate-methacrylic acid copolymers, octyl acrylate-styrene copolymers, octyl acrylate-vinyl acetate copolymers, hydroxypropyl acrylate-butylaminoethyl methacrylate-acrylic acid acrylamide copolymers, butyl acrylate-ethyl hydroxymethacrylate copolymers, butyl acrylate-hydroxymethacrylic acid copolymers, butyl acrylate-methyl methacrylate copolymers, butyl acrylate-methacrylic acid copolymers, butyl acrylate-vinyl acetate copolymers; methyl acrylate-ethyl acrylate copolymers, methyl acrylate-styrene copolymers, methoxyethyl acrylate-hydroxyethyl acrylate-butyl acrylate copolymers, methoxyethyl acrylate-hydroxyethyl acrylate copolymers, acrylic resin alkanolamines, methacrylic acid-styrene copolymers, methacrylic acid-butyl methacrylate copolymers, methacrylic acid-methyl methacrylate copolymers, methyl methacrylate-butyl acrylate-octyl acrylate copolymers, and the like, and combinations thereof.

Non-limiting examples of suitable celluloses for use in accordance with the sealer of the present disclosure include film-forming polymer, methyl cellulose, ethyl cellulose, cationized cellulose, carboxymethyl cellulose, hydroxyethyl cellulose, hydroxypropyl cellulose, hydroxypropylmethyl cellulose, etc., and combinations thereof.

Other non-limiting examples of suitable polymers for use in the sealer in accordance with the present disclosure include poly(vinyl methyl ether), vinyl methyl ether-ethyl maleate copolymers, vinyl methyl ether-butyl maleate copolymers, styrene-methylstyrene-indene copolymers, toluenesulfonamide resins, polyamide epichlorohydrin, polyethylene-imine, polyethylene glycol-epichlorohydrin-coconut oil alkylamine-dipropylenetriamine condensates, polyvinyl acetal diester aminoacetate, polyvinyl acetal diethylamino-acetate, poly(dimethylmethylenepieridinium chloride), methoxyethylene-maleic anhydride copolymers, dimethyldiallylammonium chloride-acrylamide copolymers, hydrogenated styrene-methylstyrene-indene copolymers, maleic anhydride-diisobutylene copolymer sodium salts, polyvinyl alcohol, polyvinyl butyrate, polyvinyl chloride, vinyl acetate-crotonic acid copolymers, vinyl acetate-styrene copolymers, butadiene-acrylonitrile copolymers, and the like, and combinations thereof.

In embodiments, polymer such as a film-forming polymer is added in an amount of about 3% to about 30% of the total weight of the sealer composition. In some embodiments, at least one film-forming polymer includes polyacrylic acid and/or methacrylic acid copolymer in an amount of about 5% to about 20% of the total weight of the sealer.

Solvents useful for preparing sealer solutions include any solvent capable of solubilizing suitable polymers including film-forming polymers. Such solvents include solvents capable of solubilizing natural polymers, acrylic resins, silicones, celluloses, alkyd resins, carboxyvinyl polymers, vinylpyrrolidone-based polymers, methacrylic acid copolymer, and combinations thereof. In some embodiments, such solvents include water, short chain alkyl esters, ethers, aldehydes, ketones, alcohols, and combinations thereof. In one embodiment, the sealer includes a low molecular weight solvent system of acetone and ethanol for solubilizing the film-forming polymer. In embodiments, solvents suitable for solvating anionic copolymers based on methacrylic acid and methyl methacrylic are suitable for use in accordance herein including methanol, ethanol, aqueous isopropyl alcohol, acetone, and combinations thereof.

In embodiments, solvent is added in an amount of about 30% to about 96% of the total weight of the sealer. In some embodiments, solvent includes acetone and ethanol in an amount of about 50% to about 90% of the total weight of the sealer. In some embodiments, solvent includes acetone and/or ethanol in an amount of about 50% to about 90% of the total weight of the sealer.

Penetration aids suitable for use in the sealer in accordance with the present disclosure include any penetration aid capable of disruption the barrier function of the skin. Non-limiting examples of suitable penetration aids include enzymes, keratolytic agents, acids, surfactants, DMSO, 1-dodecylazacycloheptan-2-one (available as Azone from the Upjohn Co.) and the like, 2-hydroxybenzoic acid, and combinations thereof. Penetration aids may be added to the sealer in an effective amount to disrupt the barrier function of the skin. In embodiments, the sealer solution may include penetration aid in an amount of about 5% to about 20% of the total composition. Suitable amounts of salicylic acid or 2-hydroxybenzoic acid include an amount of about 10% to about 20% by weight of the total formulation.

In preparing sealer compositions in accordance with this disclosure, film-forming agent and/or polymer is simply mixed with the disclosed solvents, at room temperature or other suitable temperature. In embodiments, at least one penetration aid is added to the sealer mix. In embodiments, the sealer is characterized as a solvent based polymer solution.

In some embodiments, sealer may be applied in a predetermined amount to skin having a predetermined amount of active solution product previously applied thereto. In embodiments, the sealer dries on the skin surface of the skin forming a film which seals the remaining drug in place. Drug trapped under this film will not be readily removed from the skin surface and therefore can still be absorbed. Another benefit is that this film acts like a bandage to protect skin surface from further environmental infections.

In some embodiments, the sealer may be any suitable film for covering a treated area on human skin. One useful embodiment contains sealer having constituents selected from the following: polyacrylic acid, methacrylic acid copolymer, salicylic acid, ethanol, methanol, isopropyl alcohol, acetone, cellulose ether, hydroxypropylcellulose, and combinations thereof.

In embodiments, the sealer may further include at least one solvent, at least one film-forming polymer, and combinations thereof. In some embodiments, the at least one solvent includes acetone and ethanol in an amount of about 50% to about 90% of the total weight of the sealer. In some embodiments, the at least one solvent includes acetone and/or ethanol in an amount of about 50% to about 90% of the total weight of the sealer. In embodiments, the at least one film-forming polymer comprises polyacrylic acid and/or methacrylic acid copolymer in an amount of about 5% to about 20% of the total weight of the sealer.

The products formulated with the present solutions can be packaged in any type of container within the purview of those skilled in the art, including, but not limited to bottles, tubes, pump type, roll-ons, daubers, wipes, and the like.

During treatment in accordance with the present disclosure active agents and formulations containing them may be topically applied to skin in need of improvement in amounts sufficient to reduce or eliminate undesirable skin conditions. As used herein the word “treat,” “treating” or “treatment” refers to using the active agent or drug compositions and/or formulations of the present disclosure prophylactically to prevent outbreaks of any undesirable skin condition, and/or therapeutically to ameliorate or cure an existing undesirable skin condition. A number of different treatments are now possible, which reduce and/or eliminate undesirable skin conditions.

As used herein “undesirable skin condition” refers to any detectable skin disorder. Such disorders can appear due to a number of factors such as, for example, chronological aging, environmental damage, and/or other diseased or dysfunctional state. Non-limiting examples of such skin disorders include the development of bacterial infection, fungal infection, viral infection, and/or parasitic infection. Other skin disorders include dryness, itchiness, thinning, thickening, wrinkling, including both fine superficial wrinkles and coarse deep wrinkles, skin lines, crevices, bumps, large pores, scaliness, flakiness and/or other forms of skin unevenness or roughness, hyperpigmentation, mottled appearance, decreased healing times, cherry angioma, telangietasias, senile development, actinic purpura development, seborrheic keratoses, actinic keratoses, fatty tissue formation, fatty tissue deterioration, increased collagen, elastin, tropoelastin, and elastic fiber content, decreased collagen, elastin, tropoelastin or elastic fiber content, and combinations thereof. Such disorders further include undesirable tactile conditions such as loss of skin elasticity, sagging, loss of skin firmness, loss of skin tightness, loss of skin recoil from deformation, and/or sallowness. Such disorders further include undesirable visible conditions such as hyperpigmented skin regions such as age spots and freckles, keratoses, abnormal differentiation, hyperkeratinization, stretch marks, discoloration, blotching, and combinations thereof. In embodiments, the skin disorder may be a crack or crevice such as those found around the heel or on the foot of a patient. In embodiments, the skin disorder may be fungal infections such as Tinea pedis, Tinea cruris, Tinea corporus, Tinea faciei, Tinea capatis, onychomycosis, and combinations thereof. It is understood, that the listed undesirable skin conditions are non-limiting and that only a portion of the skin conditions suitable for treatment in accordance with the present disclosure are listed herein.

In embodiments, compositions for use in accordance with the present disclosure contain one or more active agents in an effective amount to improve undesirable skin conditions. As used herein “effective amount” refers to an amount of a compound or composition having active agents in accordance with the present disclosure that is sufficient to induce a particular positive benefit to skin having a skin disorder. The positive benefit can be health-related, or it may be more cosmetic in nature, or it may be a combination of the two. In embodiments, the positive benefit is achieved by contacting skin with at least one antifungal agent to improve an undesirable skin condition such as a foot fungal infection. In embodiments, the positive benefit is achieved by contacting skin with one or more moisturizers to improve an undesirable skin condition such as a crack.

The particular active agent or ingredients employed, and the concentration in the compositions, generally depends on the purpose for which the composition is to be applied. For example, the dosage and frequency of application can vary depending upon the type and severity of the undesirable skin condition.

Treatments in accordance with the present disclosure contact skin with one or more active agents in amounts to improve undesirable skin conditions. In embodiments, patients are treated by topically applying an antifungal cream to skin suffering from an undesirable skin condition. In embodiments, patients are treated by topically applying to skin suffering from an undesirable skin condition, one or more moisturizers. The active agent is applied until the treatment goals are obtained. However, the duration of the treatment can vary depending on the severity of the condition. For example, treatments can last several weeks to months depending on whether the goal of treatment is to reduce or eliminate an undesirable skin condition.

In some embodiments, pharmaceutically acceptable active agent compositions relate to any formulations that contain any active agents for use in accordance with the present disclosure. For topical applications, the pharmaceutical compositions may be formulated in a suitable ointment containing the active agent suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active agents suspended or dissolved in one or more pharmaceutically acceptable carriers. Suitable carriers include, but are not limited to, mineral oil, sorbitan monostearate, polysorbate, cetyl esters wax, cetearyl alcohol, 2-octyldodecanol, benzyl alcohol and water. For example a 20% Vitamin C composition may be formulated in water and other ingredients for promoting stability, which may be topically applied to the skin.

In some embodiments, the treatments in accordance with the present disclosure can be combined with other treatments which pre-condition and/or post-condition skin in need of treatment. For example, in order to prepare the skin, a cleanser and/or toner can be applied to the treated area prior to the application of active agent and sealer.

In embodiments, the active agents and sealers are applied for cosmetic purposes only.

In some embodiments, use of a penetration aid such as 2-hydroxybenzoic acid may be included in the manufacture of a sealer for treatment of a skin condition. In such embodiments, penetration aids described in accordance with the present disclosure can be manufactured into sealer compositions. Such sealer compositions may also contain medicament, and/or formulations, and/or any excipients or ingredients described herein.

In order that those skilled in the art may be better able to practice the compositions and methods described herein, the following examples are given as an illustration of the preparation of the present compositions and methods. It should be noted that the disclosure is not limited to the specific details embodied in the examples.

### EXAMPLE 1

The benzoyl peroxide composition in Table 1 below is applied to the skin of a person having acne. The sealer composition shown below is immediately applied to the skin where the benzoyl peroxide composition is applied. The sealer forms a barrier over the treated skin preventing the benzoyl peroxide solution from being wiped off of the skin.

TABLE 1

| Ingredients | Amount |
|---|---|
| Benzoyl Peroxide Composition | |
| Water | 67.6% |
| Steareth 40 | 0.8% |
| Glycerine | 4.0% |
| Benzoyl Peroxide 74% | 7.0% |
| Benzyl benzoate | 10.0% |
| Cyclomethicone | 5.0% |
| Ethylene Diamine tetraacetic | 0.1% |
| acid disodium salt (EDTA) | |
| Stearyl alcohol | 4.0% |
| Streareth 2 | 1.5% |
| Sealer | |
| Salicylic acid | 2.0% |
| Eudagrid S100 | 1.0% |
| | (polyacrylic acid) |
| SDA 39C ethanol 200 proof | 50% |
| Hydroxypropylcellulose | 1.0% |
| Acetone | 46.0% |

### EXAMPLE 2

The benzoyl peroxide composition shown below in Table 2 is applied to the skin of a person having acne. The sealer composition shown below is immediately applied to the skin where the benzoyl peroxide composition is applied. The sealer forms a barrier over the treated skin preventing the benzoyl peroxide solution from being wiped off of the skin.

| | | RANGE OF |
|---|---|---|
| Ingredient | Amount | AMOUNTS |
| Benzoyl Peroxide | 6.25% | 2.0%-15% |
| Benzoyl benzoate | 42.45% | 20%-60% |
| Dimethyl isosorbide | 40.00% | 20%-60% |
| Vitamin E Acetate | 0.5% | 0.0%-5% |
| BHT | 0.8% | 0.0%-5% |
| Ethoxy diglycol | 10.0% | 5%-20% |
| Sealer | | |
| Salicylic acid | 2.0% | 1.0%-10% |
| Eudagrid S100 | 1.0% | 0.5%-10% |
| | polyacrylic acid | |
| SDA 39C ethanol 200 proof | 50% | 30%-70% |
| Hydroxypropylcellulose | 1.0% | 0.1%-10% |
| Acetone | 46.0% | 20%-70% |

### EXAMPLE 3

The Nystatin composition shown below in Table 3 is applied to the toes of a person having athletes foot. The sealer composition shown below is immediately applied to the skin where the antifungal composition is applied. The sealer forms a barrier over the treated skin preventing the antifungal cream from being wiped off of the skin. The sealer seals the skin surface to prevent removal of the drug or active agent. The sealer holds the drug or active agent in a reservoir film. The sealer enhances long term penetration of the drug or active agent.

TABLE 3

| Ingredient | Amount FP % |
|---|---|
| Nystatin antifungal cream | |
| Emulsifying wax, NF | 15 |
| White Petrolatum | 3 |
| Polyethylene Glycol, 300, NF | 2 |
| Propylene glycol, USP | 3 |
| Arlacel 165 | 0.5 |
| Ethoxy diglycol | 5 |
| polysorbate 60 | 0.5 |
| Isopropyl Myristate, NF | 4 |
| Sorbic Acid, NF | 0.2 |
| Glycerine, USP | 2 |
| Nystatin, USP | 1.763 |
| Deionized water | q.s. |
| Sealer | |
| Salicylic acid | 15.0% |
| Eudagrid S100 | 7.5% |
| | polyacrylic acid |
| SDA 40B ethanol 200 proof | 34.5 |
| Acetone | 43.0% |

### EXAMPLE 4

A moisturizer is applied to the heel of a person having cracked skin. The sealer composition shown below in Table 4 is immediately applied to the skin where the moisturizer composition is applied. The sealer forms a barrier over the treated skin preventing the moisturizer from being wiped off of the skin. The sealer holds the drug or active agent in a reservoir film. The sealer enhances long term penetration of the drug or active agent.

TABLE 4

| Sealer | Amount FP % |
|---|---|
| Salicylic acid | 15.0% |
| Eudagrid S100 | 7.5% |
| | polyacrylic acid |
| SDA 40B ethanol 200 proof | 34.5 |
| Acetone | 43.0% |

### EXAMPLE 5

In vitro percutaneous absorption studies were performed at the University of California Irvine (“UCI”) using radiolabeled topical benzoyl peroxide serum applied to human cadaver skin. The following protocol was followed:
Group A: Skin was wiped with a wet gauze and allowed 2 minutes to dry. 15 mg of radiolabeled benzoyl peroxide serum was applied to the skin surface and rubbed on with a rubber spatula and allowed 2 minutes to dry.
Group B: Skin was wiped with a wet gauze and allowed 2 minutes to dry. 15 mg of radiolabeled benzoyl peroxide serum was applied to the skin surface and rubbed on with a rubber spatula and allowed 2 minutes to dry. Sealer was applied and allowed to dry and form film.
Group C: Skin was wiped with a wet gauze and allowed 2 minutes to dry. 15 mg of radiolabeled benzoyl peroxide emulsion was applied to the skin surface and rubbed on with a rubber spatula and allowed 2 minutes to dry.
Results: After eight hours of application skin from Group B that utilized benzoyl peroxide serum with the sealer in accordance with the present disclosure provided 23.6% of the benzoyl peroxide applied to the stratum corneum and the epidermis. Skin from Group A, where the benzoyl peroxide serum formula was applied without the sealer enhancement provided 17.5% of the benzoyl peroxide applied to the stratum corneum and the epidermis. The control benzoyl peroxide emulsion (Group C skin) provided 10% of the benzoyl peroxide applied.

The results further showed that the benzoyl peroxide serums (where benzoyl peroxide is dissolved in a solution in Groups A and B) leave lesser amounts of product to be wiped off after eight hours than the emulsions (Group C). This finding was more dramatic when the sealer enhancement was applied on top of the serum (Group B).

Groups A and B left a greater amount of benzoyl peroxide on the surface of the stratum corneum than the emulsion of Group C.

In Group B, the treatment not only delivered the product to the skin in need thereof, but provided a reservoir of product readily available from the remaining serum.

The application of the sealer in Group B greatly improved the penetration of the benzoyl peroxide into the stratum corneum, epidermis and dermis over the compositions applied without a sealer.

It will be understood that various modifications may be made to the embodiments disclosed herein. Therefore, the above description should not be construed as limiting, but merely as exemplifications of embodiments. Those skilled in art will envision other modifications within the scope and spirit of the claims appended hereto.

What is claimed:

1. A method of improving the absorption of an active agent applied to skin comprising: topically applying an active agent to skin, and applying a sealer over the active agent.

2. The method according to claim 1 wherein the sealer comprises a penetration aid in the amount of about 5% to about 20% of the total composition.

3. The method according to claim 2 wherein the penetration aid is 2-hydroxybenzoic acid.

4. The method according to claim 1 wherein the sealer comprises a solvent and at least one polymer.

5. The method according to claim 4 wherein the polymer is selected from the group consisting of natural polymers, acrylic resins, silicones, celluloses, alkyd resins, carboxyvinyl polymers, vinylpyrrolidone-based polymers, methacrylic acid copolymer, and combinations thereof.

6. The method according to claim 4 wherein the polymer is selected from the group consisting of polyacrylic acid, poly(methyl acrylate), poly-(ethyl acrylate), poly(butyl acrylate), polyacrylamide, poly(N-isopropylacrylamide), ammonium polyacrylate, sodium polyacrylate, crosslinked sodium polyacrylate, polymethacrylic acid, poly(methyl methacrylate), poly-(ethyl methacrylate), poly(butyl methacrylate), polymethacrylamide, sodium methacrylate, acrylic acid-styrene-ammonium methacrylate copolymers, acrylic acid-styrene copolymers, acrylic acid-methacrylamide copolymers, alkyl acrylate-styrene copolymers, alkyl acrylate copolymers, ethyl acrylate-acrylamide-acrylic acid copolymers, ethyl acrylate-butyl acrylate copolymers, ethyl acrylate-ethyl methacrylate copolymers, ethyl acrylate-methyl methacrylate-acrylic acid copolymers, ethyl acrylate-methyl methacrylate copolymers, ethyl acrylate-methacrylic acid copolymers, octyl acrylate-styrene copolymers, octyl acrylate-vinyl acetate copolymers, hydroxypropyl acrylate-butylaminoethyl methacrylate-acrylic acid acrylamide copolymers, butyl acrylate-ethyl hydroxymethacrylate copolymers, butyl acrylate-hydroxymethacrylic acid copolymers, butyl acrylate-methyl methacrylate copolymers, butyl acrylate-methacrylic acid copolymers, butyl acrylate-vinyl acetate copolymers, methyl acrylate-ethyl acrylate copolymers, methyl acrylate-styrene copolymers, methoxyethyl acrylate-hydroxyethyl acrylate-butyl acrylate copolymers, methoxyethyl acrylate-hydroxyethyl acrylate copolymers, acrylic resin alkanolamines, methacrylic acid-styrene copolymers, methacrylic acid-butyl methacrylate copolymers, methacrylic acid-methyl methacrylate copolymers, methyl methacrylate-butyl acrylate-octyl acrylate copolymers, methyl cellulose, ethyl cellulose, cationized cellulose, carboxymethyl cellulose, hydroxyethyl cellulose, hydroxypropyl cellulose, hydroxypropylmethyl cellulose, poly(vinyl methyl ether), vinyl methyl ether-ethyl maleate copolymers, vinyl methyl ether-butyl maleate copolymers, styrene-methylstyrene-indene copolymers, toluenesulfonamide resins, polyamide epichlorohydrin, polyethylene-imine, polyethylene glycol-epichlorohydrin-coconut oil alkylamine-dipropylenetriamine condensates, polyvinyl acetal diester aminoacetate, polyvinyl acetal diethylamino-acetate, poly(dimethylmethylenepieridinium chloride), methoxyethylene-maleic anhydride copolymers, dimethyldiallylammonium chloride-acrylamide copolymers, hydrogenated styrene-methylstyrene-indene copolymers, maleic anhydride-diisobutylene copolymer sodium salts, polyvinyl alcohol, polyvinyl butyrate, polyvinyl chloride, vinyl acetate-crotonic acid copolymers, vinyl acetate-styrene copolymers, butadiene-acrylonitrile copolymers, and combinations thereof.

7. The method according to claim 4 wherein the solvent is selected from the group consisting of water, short chain alkyl esters, ethers, aldehydes, ketones, alcohols, and combinations thereof.

8. The method according to claim 4 wherein the solvent is selected from the group consisting of acetone, ethanol, methanol, propanol, aqueous isopropyl alcohol, and combinations thereof.

9. The method according to claim 1 wherein the sealer further comprises a penetration aid.

10. The method according to claim 9 wherein the penetration aid is selected from the group consisting of enzymes, keratolytic agents, acids, surfactants, DMSO, 1-dodecylazacycloheptan-2-one, 2-hydroxybenzoic acid, and combinations thereof.

11. The method according to claim 1, further comprising applying a cleanser to the skin.

12. The method according to claim 1, further comprising applying a toner to the skin.

13. The method according to claim 1, wherein the active agent is within a formulation.

14. The method according to claim 1 wherein the active agent is selected from the group consisting of antimicrobial compound, anti-acne compound, anti-psoriasis compound, antifungal compound, anti-eczema-compound, topical anesthetic compound, anti-inflammatory compound, vitamin, protein, anti-wrinkle composition, anti-ageing composition, whitening composition, bleaching composition, sunless tanning composition, and combinations thereof.

15. The method according to claim 1 wherein the active agent is selected from the group consisting of amino acid and amino acid derivative, insect repellant, fungicide, anti-viral agent, an anti-cancer agent, plant extract, anti-hemorrhoid compound, anti-dandruff compound, a hair-growth stimulating compound, hair loss stimulating compound, nucleic acid, anti-scabies agent, an anti-wart agent, a copper-zinc salt, and combinations thereof.

16. The method of claim 1 further comprising the step of pre-mixing an active agent with a blending composition to form a pre-mix; and applying the pre-mix to the skin.

17. The method of claim 14 wherein the percutaneous absorption of the active agent is increased compared to application of the active agent or the pre-mix without a sealer.

18. A method of treating skin with a drug or active agent comprising contacting skin with a topical formulation comprising a drug or active agent; and applying a sealer to the skin.

19. The method according to claim 18 wherein the drug or active agent is in solution form.

20. The method according to claim 18 wherein the sealer comprises a polymer system.

21. The method according to claim 18 wherein the sealer comprises constituents selected from the group consisting of polyacrylic acid, methacrylic acid copolymer, salicylic acid, ethanol, methanol, isopropyl alcohol, acetone, cellulose ether, hydroxypropylcellulose, and combinations thereof.

22. The method according to claim 18 wherein the sealer is characterized as a solvent based polymer solution.

23. The method according to claim 18 further comprising the step of drying the sealer.

24. The method according to claim 18 wherein the topical formulation comprises an additional active agent effective in treating acne.

25. The method according to claim 18 wherein the active agent is in a topical formulation characterized as a clear facial serum.

26. The method according to claim 18 wherein the active agent is in a topical formulation characterized as a stick.

27. The method according to claim 18 wherein the step of contacting skin in need of treatment thereof with a topical formulation further comprises applying a predetermined effective amount of the topical formulation.

28. A method of treating skin with an active agent comprising contacting skin with an effective amount of topical active agent; and applying a sealer to the skin, wherein the sealer comprises a penetration aid in the amount of about 5% to about 20% of the total composition.

29. The method according to claim 28 wherein the penetration aid is 2-hydroxybenzoic acid.

30. The method according to claim 28, wherein the step of applying a topical active agent comprises applying a moisturizer to cracks in the feet of a patient.

31. The method according to claim 28, wherein the step of applying a topical active agent comprises adding an antifungal to the skin of a patient.

32. The method according to claim 31 wherein the antifungal is a nystatin cream.

33. The method according to claim 28 wherein the sealer further comprises at least one solvent, at least one film-forming polymer, and combinations thereof.

34. The method according to claim 33 wherein the at least one solvent comprises acetone and ethanol in an amount of about 50% to about 90% of the total weight of the sealer.

35. The method according to claim 33 wherein the at least one film-forming polymer selected from the group consisting of methacrylic acid copolymer, polyacrylic acid, and combinations thereof in an amount of about 5% to about 20% of the total weight of the sealer.

36. Use of a penetration aid in the manufacture of a sealer composition for treatment of an undesirable skin condition.

37. The use in accordance with claim 36 wherein the penetration aid is 2-hydroxybenzoic acid.

38. The use in accordance with claim 36 wherein the undesirable skin condition is a cosmetic skin condition.
